(19) United States
(12) Patent Application Publication
(10) Pub. No.: US 2010/0323998 A1
(43) Pub. Date: Dec. 23, 2010

(54) TOPICAL COMPOSITION CONTAINING THE COMBINATION OF MUPIROCIN AND BECLOMETHASONE

(75) Inventors: Ulhas Rameshchandra Dhuppad, Nashik (IN); Vasant Sitaram Khachane, Nashik (IN); Nitin Babulal Bhamre, Nashik (IN); Prashant Dongre, Mumbai (IN); Akhilesh Dayanand Sharma, Mumbai (IN)

Correspondence Address: GLENMARK GENERICS INC., 750 CORPORATE DRIVE, MAHWAH, NJ, 07430, US

(73) Assignee: Glenmark Pharmaceuticals Ltd., Mumbai (IN)

(21) Appl. No.: 12/672,400

(22) PCT Filed: Jun. 6, 2008
(86) PCT No.: PCT/IN08/00393

§ 371 Date: Sep. 2, 2010

(30) Foreign Application Priority Data
Aug. 6, 2007 (IN) 1517/MUM/2007

(51) Int. Cl. A61K 31/573; A61P 31/04; A61P 17/00

(52) U.S. Cl. 514/171

(57) ABSTRACT

The present invention relates to a topical pharmaceutical composition comprising mupirocin and beclomethasone, a process for its preparation, its use in the treatment of infected dermatoses caused by bacteria susceptible to mupirocin and in the treatment of secondary bacterial infections in patients of steroid responsive dermatoses.

### PRIORITY

This patent application claims priority to Indian provisional Application No. 1517/MUM/2007, filed on Aug. 6, 2007, the contents of which are hereby incorporated.

### FIELD OF THE INVENTION

The present invention relates to a topical composition for the skin which contains a combination of mupirocin and beclomethasone.

### BACKGROUND OF THE INVENTION

Mupirocin is a broad-spectrum antibiotic, which can be obtained by fermentation from Pseudomonas fluorescens.

Chemically, Mupirocin (Formula I) is represented as (E)-(2S,3R,4R,5S)-5-[(2S,3S,4S,5S)-2,3-Epoxy-5-hydroxy-4-methylhexyl]tetrahydro-3,4-dihydroxy-β-methyl-2H-pyran-2-crotonic acid, ester with 9-hydroxynonanoic acid. The chemical structure is as follows:

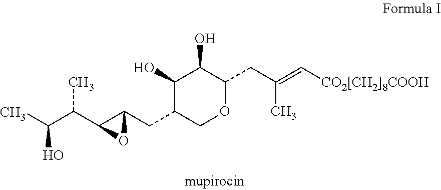

Mupirocin ointment, 2% w/w, is approved (BACTROBAN® from GLAXOSMITHKLINE) for the topical treatment of impetigo due to Staphylococcus aureus and Streptococcus pyogenes.

Mupirocin is an antibacterial agent which inhibits the growth of Gram-positive and Gram-negative bacteria. The bacteria susceptible to the in vitro action of mupirocin include aerobic strains of Staphylococcus aureus, Staphylococcus epidermidis, other Staphylococci, hemolytic α-Streptococcus positive or negative coagulase, beta hemolytic Streptococcus, Streptococcus group A (including S. pyogenes), other beta Streptococci (including S. agalactaie), Streptococcus group D (including S. faecalis and S. faecium), Streptococcus viridans group, Streptococcus pneumoniae, Corynebacterium hofmanil, Bacillus subtilis, Escherichia coli, Klebsiella pneumoniae, Proteus mirabilis, Proteus vulgaris, Enterobacter cloacae, Enterobacter aerogenes, Citrobacter freundii, Haemophilus influenzae (including producer strains of beta-lactamase), Neisseria gonorrhoeae (including producer strains of beta-lactamase), Neisseria meningitis, Branhamella catarrhalis and Pasteurella multocida, and isolated anaerobic strains of Peptostreptococcus anaerobius, Clostridium difficile and Clostridium sporogenes.

Secondary bacterial infection in skin lesions is a common problem. Bacterial infections occur frequently in lesions of eczema and atopic dermatitis. Secondary bacterial skin infections are common complications of primary dermatoses, primary nonbacterial skin infections, traumatic lesions, ulcers, cutaneous infestations, and other miscellaneous skin diseases.

Beclomethasone dipropionate (Formula II) has the chemical name 9-chloro-11b,17,21-trihydroxy-16b-methylpregna-1,4-diene-3,20-dione 17,21-dipropionate. The compound may be a white powder with a molecular weight of 521.25; and is very slightly soluble in water, very soluble in chloroform, and freely soluble in acetone and in alcohol. Its chemical structure is as follows:

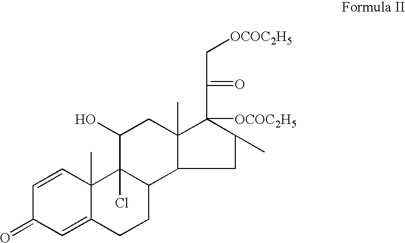

Beclomethasone dipropionate aerosol, metered inhalation is approved (QVAR® from IVAX) for the treatment of asthma.

U.S. Pat. No. 4,071,536 assigned to Glaxo describes mupirocin (previously referred as pseudomonic acid), its salts and esters.

U.S. Pat. No. 4,524,075 assigned to Beecham describes a topical composition comprising mupirocin and a stabilizer.

US 2004/0220259 assigned to AKIN describes a method of topically treating a dermatological disorder comprising topically applying a therapeutically effective amount of a cosmetic or dermatological composition to an affected area of the skin.

WO 2007/027077 assigned to UHTHOFF-ORIVE describes a composition containing a combination of mupirocin, betamethasone dipropionate, hydrogenated castor oil, polyethylene glycols and preservatives.

Sawant et al., (Journal of the Indian medical association, April 2000) undertook a study focused to determine the therapeutic efficacy and the safety of 2% mupirocin/0.05% betamethasone dipropionate ointment in the treatment of infected dermatosis. 2% mupirocin/0.05% betamethasone dipropionate ointment showed an efficiency of 94.8% in the infected dermatosis; more than 70% of these patients showed a clinical improvement after 7 days of having initiated the treatment. No adverse effects were seen during the treatment. With this data the participant doctors concluded that 2% mupirocin/0.05% betamethasone dipropionate ointment was found to be a safe and effective medicine in the treatment of infected dermatosis.

However, there is no literature in the prior art for a topical composition comprising a combination of mupirocin and beclomethasone. The inventors of the present invention formulated a topical composition containing a combination of mupirocin and beclomethasone that exhibits a synergistic effect in the treatment of infected dermatoses. Further, the combination of mupirocin and beclomethasone is also useful for the prevention of steroid-responsive dermatoses in patients who are at high risk of developing infection.

### SUMMARY OF THE INVENTION

The present invention relates to a topical pharmaceutical composition comprising mupirocin and beclomethasone. Preferably, the composition comprises mupirocin in the range of about 1-5% w/w and beclomethasone in the range of about 0.001-1% w/w (based on the total weight of the composition). For example, the composition may comprise about 2% w/w of mupirocin and about 0.025% w/w of beclomethasone (based on the total weight of the composition).

The composition may further contain one or more pharmaceutically acceptable excipients. According to one embodiment, the composition further comprises poly (substituted or unsubstituted alkylene) glycol or a derivative thereof and a pharmaceutically acceptable carrier. The composition can be in the form of an ointment, cream, lotion, solution or gel. Suitable pharmaceutically acceptable excipients for the topical composition include, but are not limited to, solvents, vehicles, ointment/cream bases, emulsifiers, preservatives, buffers, emollients, humectants, surfactants, and transport enhancers or mixtures there of.

Another embodiment is a process for preparing a topical composition comprising mupirocin and beclomethasone. The process comprises i) melting and mixing together one or more solid or semi-solid vehicles, ii) adding mupirocin and beclomethasone to the mixture of step (i) while stirring, and iii) mixing until the temperature of the composition drops below about 40° C. According to one embodiment, the one or more solid or semi-solid vehicles includes poly (substituted or unsubstituted alkylene) glycol or a derivative thereof.

Another embodiment is a method for the treatment of infected dermatoses caused by bacteria susceptible to mupirocin in a patient in need thereof comprising administering an effective amount of a composition comprising mupirocin and beclomethasone to the patient. Preferably, the composition is topically applied.

Yet another embodiment is a method for the treatment or prevention of secondary bacterial infections in a patient having a steroid-responsive dermatosis comprising administering an effective amount of a composition comprising mupirocin and beclomethasone to the patient. According to one embodiment, the dosage strength of mupirocin is in the range of 1-5% w/w, and the dosage strength of beclomethasone is in the range of 0.005-1% w/w based on the total weight of the composition.

Yet another embodiment is the use of mupirocin and beclomethasone in the preparation of a medicament for the treatment of infected dermatosis caused by bacteria susceptible to mupirocin. Yet another embodiment is the use of mupirocin and beclomethasone in the preparation of a medicament for the treatment or prevention of secondary bacterial infections in a patient having steroid-responsive dermatosis.

### DETAILED DESCRIPTION OF THE INVENTION

The formulation of the present invention comprises mupirocin in combination with beclomethasone. The formulations of the present invention are topical and may be in the form of an ointment, cream, lotion, or gel which can apply on affected skin surface.

The term “beclomethasone” as used herein includes beclomethasone free base and its pharmaceutically acceptable salts or esters thereof.

The term “mupirocin” as used herein includes mupirocin free base and its pharmaceutically acceptable salts or esters thereof.

As used herein the term “poly(substituted or unsubstituted alkylene)glycol” refers to polymers having the repeating units —(CH₂)ₙO— wherein n is an integer, preferably 2 or 3, wherein one or more methylene groups of each repeating unit is optionally substituted, and to derivatives thereof. Such polymers are known by a variety of names, for instance when n=2, as polyethylene glycol, polyoxyethylene, polyoxyethylene glycol and macrogol; and, when n=3, as polypropylene glycol, polyoxypropylene and polyoxypropylene glycol.

Suitable substituents include alkoxy groups, such as methoxy, as in polymethoxypropylene glycol.

Suitable derivatives include cross-linked polyethylene glycol and ethers and esters of poly(substituted or unsubstituted alkylene) glycols, such as macrogol ethers and esters (e.g., cetomacrogol), glycofurol, the ‘Tweens’ (e.g., Tween® 20 and Tween® 80), and block copolymers that include poly(substituted or unsubstituted alkylene)glycols, such as Poloxamers (block copolymers of polyethylene glycol and polypropylene glycol, e.g., the ‘Pluronics’).

According to one embodiment, polyethylene glycol or a derivative thereof with a molecular weight ranging from 200 to 8000 daltons is preferably used. Polyethylene glycols (PEGS) and derivatives are commercially available in a variety of chain lengths and with a variety of consistencies like liquids, semisolids, and hard solids. They can be used alone or as a combination of different grades and types of polyethylene glycols with different molecular weight as principal vehicles and/or as thickeners.

Formulations of the present invention may be produced by conventional pharmaceutical techniques. Thus, ointments and creams are conveniently prepared by melting and mixing together the solid or semi-solid vehicles, and stirring in the therapeutic agent and any other ingredients. The product is then slowly cooled and filled into containers such as collapsible metal or plastic tubes.

The term “composition” includes suitable dosage forms, such as ointments, creams, lotions, gels or solutions, and may contain appropriate conventional additives, such as preservatives, solvents and emollients, cream or ointment bases, viscosity modifiers, stiffening agents, emulsifiers, preservatives, buffers, vehicles and mixtures there of. Such carriers may comprise from about 1% to about 98% of the formulation. Preferably, carriers will form up to about 80% of the formulation.

Ointment bases include, but are not limited to, Polyethylene glycols (PEGS), Bis-Diglyceryl Polyacyladipate-2 (Softisan 649), paraffins and mixtures there of.

Stiffening agents include, but are not limited to, fatty alcohols or esters such as stearyl alcohol, cetyl alcohol, myristyl alcohol, cetyl stearyl alcohol, glycerin monostearate and mixtures there of. Preferably, they are used in the range from 5-10%.

Emulsifiers include, but are not limited to, polyoxyethylene glycol monocetyl ethers, such as the material sold under the trade name cetomacrogol 1000, and polyoxyethylene sorbitan monostearates, such as the material sold under the trade name Polysorbate 60, or polyoxyethylene sorbitan monoleates, as sold under the trade name Tween 80, sorbitol monostearate (Span 60), glyceryl monostearate and mixtures there of. Preferably, they are used in the range from 4-10%.

Emollients include, but are not limited to, 2-Octyldodecanol, “mineral oil” and mixtures there of. Preferably, they are used in the range from 5-10% w/w. The term “mineral oil” as used herein includes any that is suitable for use in a topical pharmaceutical composition and includes mineral oil USP, light mineral oil NF, liquid paraffin BP and light liquid paraffin BP.

As used herein, treatment means the prophylaxis, prevention or amelioration of one or more symptoms of, or associated with secondary and primary bacterial infection.

Preferably, mupirocin in the range of 1-5% w/w, and beclomethasone in the range of 0.001-1% w/w (based on the total weight of the composition) are used. More preferably, mupirocin 2% w/w and beclomethasone dipropionate 0.025% w/w (based on the total weight of the composition) are used.

The topical composition of the present invention can be used for the treatment of infected dermatoses caused by bacteria susceptible to mupirocin. This includes the treatment of lesions like eczema, atopic dermatitis, allergic dermatitis, contact dermatitis, and psoriasis, (which are primarily inflammatory in nature and responsive to treatment with corticosteroids) that have been further infected by bacterial infection which signifies secondary bacterial infection. It may also be used for the prevention of steroid responsive dermatoses in patients who are at high risk of developing infection.

The topical composition can be used to prevent the exacerbation of steroid-responsive dermatoses. In order to reduce the risk of inducing antibacterial drug resistance, administration of mupirocin and beclomethasone for a period of 1 to 2 weeks may alternate with administration of a topical steroid alone.

The invention is further illustrated by the following examples, which are not intended to limit the scope of the invention.

### Example 1

| S. No. | Ingredients | % w/w |
|---|---|---|
| 1 | Mupirocin | 2.0 |
| 2 | Beclomethasone Dipropionate | 0.025 |
| 3 | Macrogol 400 | 70.975 |
| | (PEG 400/Polyethylene Glycol 400) | |
| 4 | Macrogol 4000 | 27.00 |
| | (PEG 4000/Polyethylene Glycol 4000) | |

10% overages are added.

### 1. Preparation of Mupirocin and Beclomethasone Dipropionate Phase

1.1. Macrogol 400 (Polyethylene Glycol 400) was heated up to about 40° C. to 70° C. with the aid of steam with frequent stirring.
1.2. Mupirocin and beclomethasone dipropionate were added to (step 1.1) at about 40° C. to 70° C. under stirring (Homogenises), and stirring was continued for 8 to 10 minutes to form a clear solution.

### 2. Macrogol 4000 (Polyethylene Glycol 4000) Melting Phase

Macrogol 4000 (PEG 4000) and macrogol 400 (PEG 400) (from step 1) were added and heated to about 40° C. to 70° C. with aid of steam in a jacketed planetary mixer bowl, and the mixture was stirred frequently to from a clear phase.

### 3. Mixing

3.1. Contents of (step 2) were added to contents of (step 1) by filtering through 100# nylon cloth at 40° C. to 70° C.
3.2. The mixture was stirred at slow speed (18 rpm) in a planetary mixer. After 15 minutes, tap water was circulated through jacket for cooling. Mixing was continued until the temperature of the composition dropped below 40° C.

### Example 2

| Steps | Ingredients | % w/w | CATEGORY |
|---|---|---|---|
| I | Bis-Diglyceryl Polyacyladipate-2 | 20.00 | Ointment base |
| | (Softisan 649) | | |
| | White Soft Paraffin q.s. | 77.975 | Ointment base |
| II | Beclomethasone Dipropionate, | 0.025 | Drug |
| | micronised Mupirocin, micronised | 2.000 | Drug |
| | | 100 | |

### Brief Manufacturing Process

### 1. Oil Phase

Bis-Diglyceryl Polyacyladipate-2 was heated up to about 40° C. to 70° C. with frequent stirring.

### 2. Drug Phase

Both the drugs were dispersed in (step 1) at 55° C. to 58° C.

3. Mixing and homogenization

The contents of (step 2) were homogenized for about 10 minutes.

4. Mixing was continued until the temperature of the composition dropped to room temperature.

### Example 3

### Clinical Study

A study was conducted to compare the efficacy, safety and tolerability of mupirocin 2%+beclomethasone dipropionate 0.025% ointment in comparison with beclomethasone dipropionate 0.025% ointment for prevention of Secondary Bacterial Infections.

### The Study Protocol was as Follows:

Trial Design: A prospective, Randomized, Double Blind, comparative study.
Duration of study: 2 weeks.
Number of Patients: 40 in each group

### Number of Centres: One

### Study Medication:

The patients were asked to apply the mupirocin 2%+beclomethasone dipropionate 0.025% ointment or beclomethasone dipropionate 0.025% ointment to the affected area three times daily for a period of 2 weeks.

### Trial Criteria

Inclusion Criteria
a) Male or female patients of age 18 to 65 yrs.
b) Clinical diagnosis of steroid responsive dermatoses but not having any overt bacterial or fungal infection.
c) Written informed consent by patients.
d) Patient willing to follow up.

Exclusion Criteria
a) Patient not willing to participate in the trial or not in position to give the informed consent.
b) Patient known to have hypersensitivity to either mupirocin 2% w/w or Beclomethasone 0.025% w/w.
c) Pregnant or lactating females.
d) Patients who have already received local or systemic antibiotic in the last 24 hours prior to inclusion.
e) Patients having severe infection requiring systemic antibiotic, in the opinion of the treating dermatologist.
f) Patients with other co-existing severe medical disease.

Treatment Groups:

Group A: patients treated with steroid (beclomethasone) alone for 2 weeks.
Group B: patients treated with mupirocin and beclomethasone for 2 weeks.

### Efficacy Parameters

The patient will be examined at the time of inclusion in the study. After getting written informed consent, patient will be entered in to the study.

1. Increase in the signs and symptoms.

The signs and symptoms included: Itching, pain/tenderness, erythema, exudation and crusting, scaling, lichenification.

Efficacy: based on changes in sign & symptoms from the start of trial to end of trial, i.e., Day 14 with including follow-up period of Day 3 and 7.

The dermatologists were requested to rate the patient on five different signs and symptoms as ‘0’=Absent, ‘1’=Mild, ‘2’=Moderate and ‘3’=Severe, before and after the treatment. The signs and symptoms included: Itching, pain/tenderness, erythema, exudation, crusting, scaling, lichenification.

2. Colony count of staphylococcus aureus before the start of the study and after 2 weeks of treatment.

### Results of the Study:

COMPARISON OF CHANGES IN MEAN SCORE
OF ITCHING BETWEEN TWO GROUPS.

| | Mean Score ( X̅ ± SD) | |
|---|---|---|
| | Mupirocin 2% + | |
| | Beclomethasone | Beclomethasone |
| | Dipropionate 0.025% | Dipropionate 0.025% |
| Duration in days | ointment | ointment |
| Basal | 2.11 ± 0.97 | 2.06 ± 1.14 |
| 3 | *1.15 ± 0.63 @ | *1.52 ± 0.87 |
| 7 | *0.41 ± 0.59 | *0.84 ± 0.69 |
| 14 | *0.18 ± 0.24 | *0.48 ± 0.32 |

By ANOVA (as determined using Kruskal Walli's test)
*P < 0.05 Vs Basal, Significant
@Between Groups P < 0.05 Significant

The above table shows that mean score of Itching was 2.11 among the case who used mupirocin+beclomethasone dipropionate and 2.06 in other group at basal, a statistically insignificant difference.

After the treatment, at 3^{rd} day mean score showed a statistically significant decrease of 45.5% among mupirocin+beclomethasone dipropionate group as compared to 26.2% in beclomethasone dipropionate group.

At the end of 7^{th} and 14^{th} day, the reduction in mean score was 80.6% and 91.5% among mupirocin+beclomethasone dipropionate group as compared to 59.2% and 76.7% among other group.

COMPARISON OF CHANGES IN MEAN SCORE
OF ERYTHEMA BETWEEN TWO GROUPS

| | Mean Score ( X̅ ± SD) | |
|---|---|---|
| | Mupirocin 2% + | |
| | Beclomethasone | Beclomethasone |
| | Dipropionate 0.025% | Dipropionate |
| Duration in days | ointment | 0.025% ointment |
| Basal | 2.54 ± 1.36 | 2.63 ± 1.41 |
| 3 | *1.37 ± 0.88 @ | *1.88 ± 1.01 |
| 7 | *0.61 ± 0.49 @ | *0.94 ± 0.68 |
| 14 | *0.20 ± 0.38 @ | *0.60 ± 0.51 |

By ANOVA (as determined using Kruskal Walli's test)
*P < 0.05 Vs Basal, Significant.
@ Between Groups P < 0.05 Significant

Mean score of erythema was 2.54 and 2.63, respectively, in Mupirocin+Beclomethasone Dipropionate and Beclomethasone Dipropionate groups at basal, a difference which is not statistically significant.

After the treatment, at the end of 7^{th} day, mean erythema was significantly decreased in both the groups. At the end of 3^{rd} day, the decrease was 46.1% in Mupirocin+Beclomethasone Dipropionate and 28.5% among Beclomethasone Dipropionate.

After the end of treatment, mean erythema score exhibited a decrease of 92.1% and 77.2%, respectively, in mupirocin+beclomethasone dipropionate and beclomethasone dipropionate groups.

COMPARISON OF CHANGES IN MEAN SCORE
OF SCALING BETWEEN TWO GROUPS

| | Mean Score ( X̅ ± SD) | |
|---|---|---|
| | Mupirocin 2% + | |
| | Beclomethasone | Beclomethasone |
| Duration in days | Dipropionate 0.025% | Dipropionate 0.025% |
| Basal | 1.12 ± 0.95 | 1.24 ± 0.88 |
| 3 | *0.66 ± 0.82 @ | *0.72 ± 0.75 |
| 7 | *0.24 ± 0.39 @ | *0.43 ± 0.59 |
| 14 | *0.10 ± 0.19 @ | *0.28 ± 0.33 |

By ANOVA (as determined using Kruskal Walli's test)
*P < 0.05 Vs Basal, Significant.
@ Between Groups P < 0.05 Significant

The above table shows that mean score of scaling was 1.12 and 1.24 respectively in mupirocin+beclomethasone dipropionate and beclomethasone dipropionate group at basal.

After the treatment, at the end of 3^{rd} day, the mean dryness score had decreased significantly in both the group, i.e., 41.1% among mupirocin+beclomethasone dipropionate group and 41.9% in Beclomethasone Dipropionate group.

After the end of 14^{th} day, the reduction was 91.1% in the mupirocin+beclomethasone dipropionate group compared to 77.4% in the beclomethasone dipropionate group.

PROFILE OF BACTRERIOLOGICAL FINDINGS

| | Mupirocin 2% + | | | |
|---|---|---|---|---|
| | Beclomethasone | | Beclomethasone | |
| | Dipropionate 0.025% | | Dipropionate 0.025% | |
| | (N = 39) | | (N = 38) | |
| | Before | After | Before | After |
| Bacterial | treatment | treatment | treatment | treatment |
| isolates | No. (%) | No. (%) | No. (%) | No. (%) |
| No growth | 03 (7.7) | *37 (94.9) | 04 (10.5) | @ *27 (72.3) |
| Significant | 36 (92.3) | 02 (05.1) | 34 (89.5) | 11 (27.7) |
| growth | | | | |
| Staph. Aureus | 25 (64.1) | 01 (02.6) | 22 (57.9) | 07 (18.4) |
| Staph. | 04 (10.3) | — (—) | 05 (13.2) | 02 (05.3) |
| epidermidis | | | | |
| Streptococcus | 02 (05.1) | — (—) | 03 (07.9) | 01 (02.6) |
| spp. | | | | |
| Entero- | 04 (10.2) | 01 (02.6) | 03 (07.9) | 01 (02.6) |
| bacteriace | | | | |
| Misc* | 01 (02.6) | — (—) | 01 (02.6) | — (—) |

By chi - Square Test
*P < 0.05 Significant
*Pseudomonas
@ Between Group
P < 0.05 Significant

89.5-92.3% of the total cases had significant bacteriological growth in both the groups at baseline. After treatment, only 5.1% of the cases had a significant growth among the mupirocin+beclomethasone dipropionate group and 27.7% among the beclomethasone dipropionate group. At the same time, 94.9% of total cases exhibited no bacterial growth among the mupirocin+beclomethasone dipropionate group, while only 72.3% saw no growth in the beclomethasone dipropionate group.

PROFILE OF ADVERSE EVENTS

| | Mupirocin 1% + | | | |
|---|---|---|---|---|
| | Beclomethasone | | Beclomethasone | |
| | Dipropionate 0.025% | | Dipropionate 0.025% | |
| | (N = 40) | | (N = 40) | |
| Adverse Events | No. | % | No. | % |
| Irritation | 01 | 2.5 | 02 | 5.0 |
| Itching | 02 | 5.0 | 03 | 7.5 |
| Burning Sensation | 02 | 5.0 | 01 | 2.5 |
| No. of Pts | 03 | 7.5 | 04 | 10.0 |

By Chi - Square Test
P > 0.05 Not Significant

7.5-10.0% of total cases suffered an adverse event, with no statistically significant difference between the mupirocin+beclomethasone dipropionate group and the beclomethasone dipropionate group. The most common events were itching followed by irritation and burning sensation in both the groups. The intensity of events was mild to moderate in both the groups, which were dissolved during the treatment.

### Conclusion:

This study shows that in patients of steroid responsive dermatoses, the fixed dose combination of mupirocin 2% and beclomethasone showed better and faster resolution of symptoms of dermatoses as compared to beclomethasone used alone. The bacteriological findings also showed significantly less secondary bacterial infections (primarily staphylococci) in the combination group as compared to the steroid alone. Thus, it proves that the combination of mupirocin and beclomethasone is useful in preventing the secondary bacterial infections in steroid responsive dermatoses and hence earlier resolution of the symptoms than steroid alone.

### Example 4

A study was conducted to compare the evaluation of efficacy, safety and tolerability of supirocin-B ointment (mupirocin 1%+beclomethasone 0.005%) vs. mupirocin ointment 1% in the treatment of infected dermatoses. The results of which are outlined below:

### Background and Abstract of the Study:

The aim of this study was to comparatively assess the efficacy, safety and tolerability of Supirocin-B Ointment (Mupirocin 1%+Beclomethasone 0.005%) in comparison with Mupirocin 1% in patients suffering from infected dermatoses.

### Methods:

A prospective, open, comparative, post-marketing study was undertaken in 94 patients suffering from various infected dermatoses. The patients were asked to apply the Supirocin-B Ointment or Mupirocin 1% to the affected area three times daily for a period of 2 weeks. The patients were assessed for the improvement in signs and symptoms like itching, pain, erythema, exudation, crusting, scaling and lichenification. Efficacy was also evaluated by the investigators' global improvement scale at the end of the study. Safety and tolerability was assessed on the basis of physical examination and monitoring of treatment emergent adverse events.

### Results:

A total of 92 patients (47 in the mupirocin and beclomethasone group and 45 in the mupirocin group) with infected dermatoses completed the study. A significant improvement was seen in all the signs and symptoms of itching, erythema, pain/tenderness, crusting, exudation, scaling and lichenification in the mupirocin and beclomethasone group from as early as third day up to the end of the study. 78.7% of cases had very good to excellent improvement in the investigators' global improvement scale in the mupirocin and beclomethasone group, compared to 60.0% among the mupirocin group, the difference being statistically significant. The adverse effects were mild to moderate irritation and burning sensation, and the difference between the groups was not statistically significant.

### Conclusion:

The results of the above clinical study suggest that the mupirocin and beclomethasone combination ointment is more effective than mupirocin alone in the treatment of infected dermatoses.

We claim:

1. A topical pharmaceutical composition comprising mupirocin and beclomethasone.

2. The topical pharmaceutical composition according to claim 1, wherein said composition is an ointment, cream, lotion, solution or gel.

3. The topical pharmaceutical composition according to claim 1 or 2, further comprising poly (substituted or unsubstituted alkylene) glycol or a derivative thereof and a pharmaceutically acceptable carrier.

4. The topical pharmaceutical composition according to any of claims 1-3, comprising mupirocin in the range of about 1-5% w/w and beclomethasone in the range of about 0.001-1% w/w based on the total weight of the composition.

5. The topical pharmaceutical composition according to claim 4, wherein mupirocin content is about 2% w/w and beclomethasone content is about 0.025% w/w based on the total weight of the composition.

6. The topical composition according to any of the preceding claims, further comprising solvents, vehicles, ointment/cream bases, emulsifiers, preservatives, buffers, emollients, humectants, surfactants, and transport enhancers or mixtures there of.

7. A process for preparing a topical composition comprising:
i) melting and mixing together one or more solid or semi-solid vehicles;
ii) adding mupirocin and beclomethasone to the mixture of step (i) while stirring; and
iii) mixing until the temperature of the composition drops below 40° C.

8. The process according to claim 7, wherein the one or more solid or semi-solid vehicles comprises poly (substituted or unsubstituted alkylene) glycol or a derivative thereof.

9. Use of mupirocin and beclomethasone in the preparation of a medicament for the treatment of infected dermatoses caused by bacteria susceptible to mupirocin.

10. Use of mupirocin and beclomethasone in the preparation of a medicament for the treatment or prevention of secondary bacterial infections in patients of steroid responsive dermatoses.

11. Use of a pharmaceutical composition according to any of claims 1-6 for the treatment of infected dermatoses caused by bacteria susceptible to mupirocin.

12. Use of a pharmaceutical composition according to any of claims 1-6 for the treatment or prevention of secondary bacterial infections in patients of steroid responsive dermatoses.

13. A method for the treatment of infected dermatoses caused by bacteria susceptible to mupirocin comprising administering an effective amount of a composition comprising mupirocin and beclomethasone.

14. A method for the treatment or prevention of secondary bacterial infections in patients of steroid-responsive dermatoses comprising administering an effective amount of a composition comprising mupirocin and beclomethasone.

15. The method according to claim 13 or 14, wherein the dosage strength of mupirocin is in the range of 1-5% w/w, and the dosage strength of beclomethasone is in the range of 0.005-1% w/w based on the total weight of the composition.

16. The method according to claim 15, wherein the dosage strength of mupirocin is about 2% w/w and the dosage strength of beclomethasone is about 0.025% w/w based on the total weight of the composition.
